(19) United States
(12) Patent Application Publication
(10) Pub. No.: US 2004/0186488 A1
(43) Pub. Date: Sep. 23, 2004

(54) Method of peripheral nerve reconstruction using a micro suction connector

(76) Inventor: Karl William Droese, Big Bend, WI (US)

Correspondence Address: KARL W. DROESE, S99 W24280 FOREST HOME AVE., BIG BEND, WI, 53103, US

(21) Appl. No.: 10/388,562

(22) Filed: Mar. 17, 2003

(51) Int. Cl. A61B 17/08

(52) U.S. Cl. 606/152

(57) ABSTRACT

Disclosed is a method of peripheral nerve or blood vessel reconstruction requiring the use of a unique connector. The method employs negative gauge pressure, applied through a port on the connector, to draw the ends of the disrupted nerve or vessel into the connector. Next a biocompatible adhesive is used to cement near the ends of the nerve or vessel circumferentially to the inside of the connector wall, leaving the cut ends touching each other but free of the bio-adhesive. After the placement of the bio-adhesive, additional suction is applied to a port in the temporary housing surrounding the porous connector. This draws the nerve or blood vessel to the full diameter of the connector, maximizing the functionality of a healing blood vessel, providing alignment for disrupted tissue, and improving the circulation of blood around a regenerating nerve.

### CROSS REFERENCE TO RELATED APPLICATIONS

Not Applicable

### COPYRIGHT STATEMENT

Not applicable

### FEDERAL RESEARCH STATEMENT

Not Applicable

### APPENDIX DATA

Not Applicable

### BACKGROUND OF INVENTION

Surgical repair of injuries to peripheral nerve tissue may be indicated when damage to the nerve is severe and spontaneous regeneration is unlikely or entirely precluded. The anatomy of the peripheral nervous system can be largely divided into two groups of cells. The first type of cell is directly involved with the transmission of the neural impulse signal and is called neurons. A neuron typically consists of a cell body near one end, a synaptic terminal at the other, and an interconnecting axon. A signal is transmitted using an ionic current. This process is referred to as the propagation of an action potential.

The other group of cells in the peripheral nervous system is collectively called glial cells, which include of a variety of specific cell types that provide support for the neurons. These cells offer various forms of support. Schwann cells are wrapped around the axon and insulate the progagation of the action potential. Other cells in this group provide nutrients, physical protection, and immunological defense. The Schwann cells surround the neuron and form an insulating conduit to preserve the signal or action potential traveling in the neuron. Peripheral neurons can regenerate and by using the infrastructure of the distal severed nerve, they are guided to the appropriate muscle.

There are three basic levels of injury to nerves. Neuropraxia is the mildest nerve injury. It is a reversible block in the conduction of an action potential along a neuron. The neuron remain intact and is functional elsewhere, as are the supporting cells. Recovery is spontaneous after removal of the causative agent and does not require surgery.

The intermediate level of nerve injury is called axonotmesis. The axon, or the long extension from the neuron cell body, is irreparably damaged and cannot transmit an action potential.

The supporting cells surrounding the axon are spared and provide a natural guide for the regeneration axon. This type of nerve injury also does not require surgical repair.

The most severe grade of injury damages both the neurons and the supporting cells and tissue. Without the Schwann cells, as well as the surrounding connective tissue, the damaged axon is not stimulated to regenerate. It is in this setting that surgical intervention may benefit the patient.

The goal of peripheral nerve reconstruction is to rejoin the nerve, facilitating regeneration of the proximal stump by the guiding presence of the distal part, which has filled with Schwann cells in place of the degenerated axon. The factors involved in rejoining the nerve segments include mechanically securing the nerve ends in close proximity to each other while not inhibiting the regeneration process by the same mechanical means necessary to join the ends. One technique, if the nerve is of sufficient size, is suturing the ends together. Another current technique involves gluing the nerve ends together with a biocompatible adhesive. Guide tubes impregnated with nerve growth factors have also been used to facilitate the directional growth of the axon.

Another potential use for the device and method described herein is microvasculature reconstruction. Vessels that may be too small currently to reconnect due to time constraints as well as tediousness may be candidates for repair using this device and technique.

The use of the term “microstructure” herein shall include the small nerves and the blood vessels that can be joined using this device and technique.

### SUMMARY OF INVENTION

While the use of tubes or conduits in the past has been focused on providing a conduit for the growing proximal neuron, this invention employs the connector as a structural device that immobilizes the joining area of the nerve segments. It also provides a mechanical barrier for the microenvironment around the rejoined nerve. More importantly, by using negative gauge pressure during application, it incorporates a means to draw extremely small and flexible fibers into the connector. Using both the device and the method describe herein, the efficiency and efficacy of microsurgery may be improved.

Conceptually, the device is a hollow “T” connector, where the arms of the “T” provide the conduit for the microstructure that is being repaired. The leg of the “T” is the port where suction is applied to draw in the cut ends of the microstructure. The arm walls of connector can be porous but have a temporary housing around them for the purpose of drawing the cut ends into the connector. Once the nerve ends or vessel ends have been drawn into the device, suction can be applied within the surrounding housing to expand the nerve or vessel to the full diameter of the connector. Biocompatible adhesive is used to cement the vessels or nerves in place against the inside diameter of the device.

The device provides protection of the joint as well as a rigid form to allow the microstructure to perform its normal function in the case of a blood vessel.

### BRIEF DESCRIPTION OF DRAWINGS

FIG. 1. Basic Micro Connector. This illustrates one possible geometric configuration of the device as well as a possible woven fiber method of construction.

FIG. 2. Porosity of Connector Wall. This depicts a means to provide suction during adhesion of the microstructure to the inside wall of the device.

FIG. 3. Housing for Providing Suction through the Connector Wall. The housing would effectively block the pores during the first stage of drawing the severed nerves into the

FIG. 4. One half of a 3-way connector. This shows a variation that would allow the device to be placed around an intact nerve serving as the host for a nerve graft that would be drawn into the remaining conduit.

FIG. 5. Extended Micro Connector for Graft. The device can be of any length, accommodating the placement of a graft nerve between two umbilical ports. This would facilitate repairs to damaged nerves that were too short to reconnect directly.

### BRIEF DESCRIPTION OF SEQUENCES

Not Applicable

### DETAILED DESCRIPTION

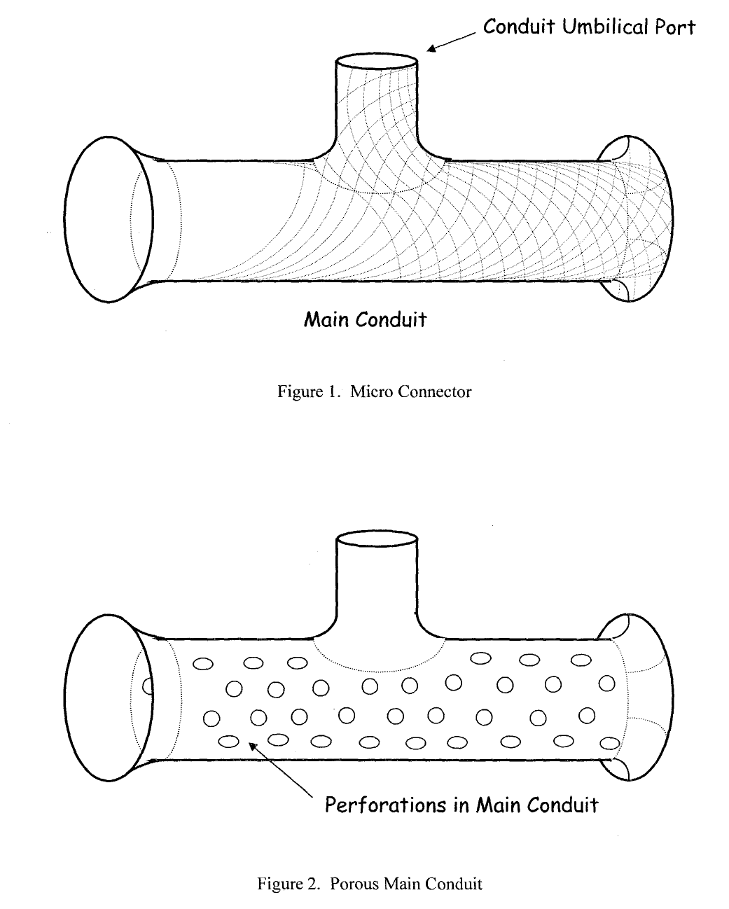

The device, in its simplest form, consists of an extracellular matrix (collagen) or other biocompatible material woven or molded into the shape of a tube with an umbilical port in the middle of the tube. (See FIG. 1.) The device is then saturated with fibrin glue or other biocompatible material to make the tube rigid, however leaving the main conduit porous. (See FIG. 2). Flaring of the ends on the main conduit would facilitate entry of the microstructures.

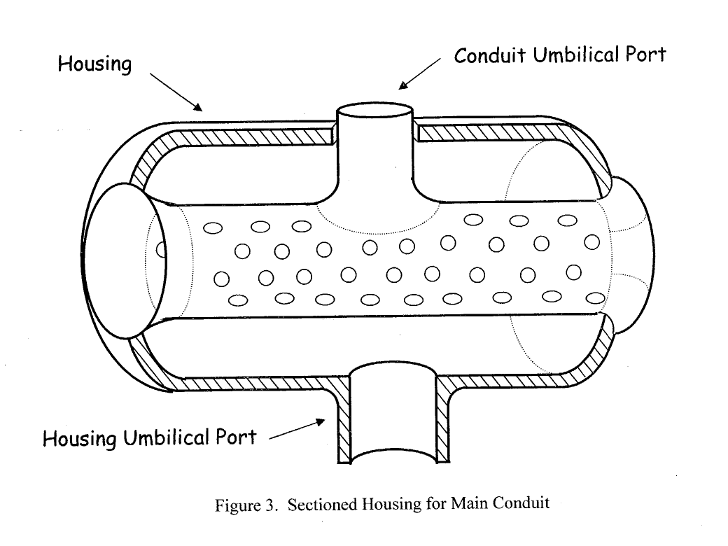

The purpose of the optional porosity of the main conduit is to allow suctioning the microstructure up against the inner wall of the conduit after an adhesive has been applied or injected into space. A housing would be placed around the device to effectively block the pores in the main conduit from atmospheric pressure, while the microstructures are initially suctioned into the device by applying suction at the umbilical port. (See FIG. 3.) After the microstructures are located within the main conduit and adhesive has been applied, suction can be applied within the housing. This expands the microstructure up against the inside of the device, which would allow for blood flow through a blood vessel, for instance. In the case of nerve repair, drawing the outer sheath of the nerve up against the inner wall of the device facilitates blood flow through the vasa nervosum, or the tiny blood vessels that surround and supply a nerve with blood.

In addition to acting as a substitute for the normal function of the microstructure, the alignment provided by the device, as the microstructures are adhered to the inner wall, facilitates healing. In the case of blood vessels, the device would limit the motion due to expansion of the vessel from the pulsating blood flow, also facilitating the healing process.

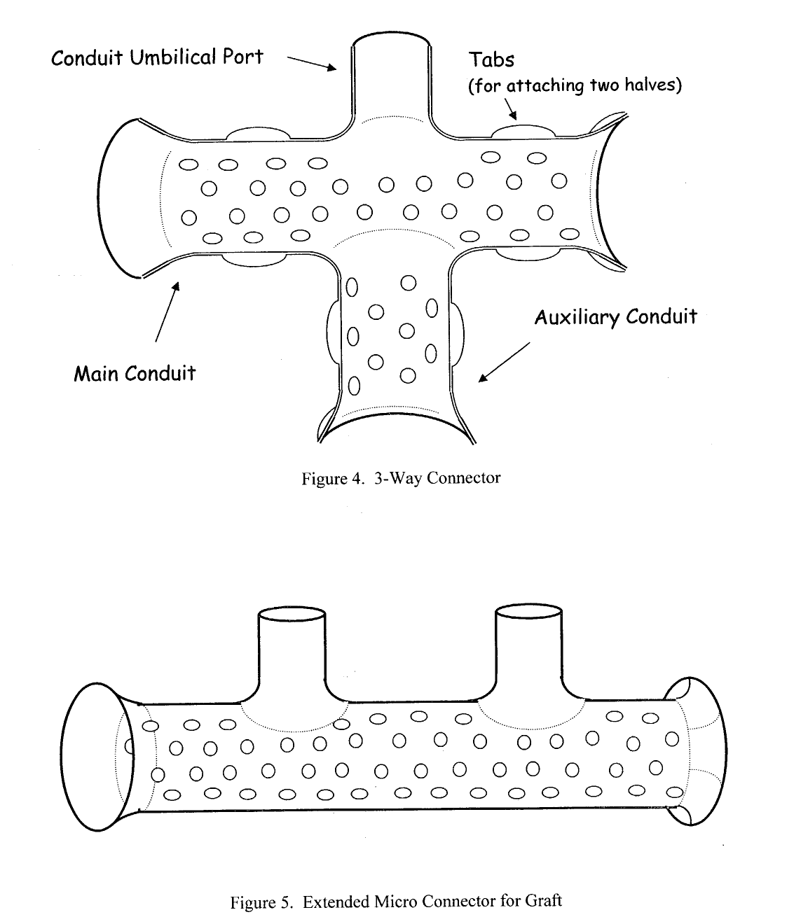

Variations in geometric form include a cross connector to allow a three way splice. The connector could be in two pieces along the plane defined by the two centerlines of the conduits, As shown in FIG. 4. It could be snapped together or glued together around the intact vessel or nerve, leaving the third and fourth ports available for the branched microstructure and suction respectively.

The tube may be impregnated with growth factors such as insulin-like growth factors, nerve growth factor, or other neurotrophins to promote axon growth in the case of neural reconstruction. In the use of blood vessel repair, other growth factors such as vascular endothelial growth factor may be considered. The potential exists for coating the exterior of the tube with a cytostatic material to inhibit fibroblastic activity near the joining sections of nerve or vessel. This would serve to limit the amount of scar tissue formed in this region. An ancillary benefit of using suction to draw the microstructures into the main conduit may be the subsequent concentration of naturally occurring growth factors at the joint between the cut ends of the microstructure due to the suctioning.

The device could also be coated with heparin or similar substance to inhibit the formation of thrombi or clots on the device. Gluing the microstructure up against the inner wall would provide a seal for blood, reducing the dependence on a thrombus to stop bleeding in the case of repairing blood vessel.

A variation in design would include more than one umbilical port on a connector whose length was extended. This would allow for placement of a microstructure graft between the umbilical ports. (See FIG. 5.) A repair could be made to a shortened microstructure by use of an interposing graft.

### DESCRIPTION OF THE METHOD

Resection of the proximal stump back to the “functioning” nerve, as determined intraoperatively, prepares the transected peripheral nerve bundle. Similarly, trimming a blood vessel back to viable tissue allows optimal conditions for healing. Suction is applied to the umbilical port as the cut ends of the microstructure are introduced at both ends of the main conduit. The suctioning approximates the ends of the microstructures. As suction is continued, with the microstructure stabilized, adhesive is introduced at the entrances of the main conduit where the microstructures enter. This provides a permanent stabilization of the microstructure within the conduit without directly coating the cut ends of the microstructure.

A variation to suctioning the adhesive into the sleeve would be to remove suction when the ends meet in the middle of the conduit. Fibrin adhesive is injected into the umbilical port, while each end of the microstructure is stabilized at the entrances of the main conduit. This alteration would coat the cut ends of the microstructure with the adhesive in addition to the area surrounding the microstructure, should the adhesive include additives to promote growth and healing.

Following the injection of the adhesive, suction is also applied to the housing that covers the porous main conduit. See FIG. 3. This draws the microstructure up against the inner wall. In the case of a repair to a blood vessel, prior to introducing the ends into the connector, the vessels could be ligated or clamped a short distance from the repair site, with the blood stripped out. This would prevent loss of vacuum by blood entering device through the blood vessel.

Suction could be applied until the adhesive sets. After suction is removed, the umbilical port can be trimmed off and a dot of adhesive applied to the opening to seal the joint. Removal of the clamps or ligature will allow inspection for blood leaks.

Program Listing Deposit

Not applicable

I claim:

1. A connecting device which includes a(n):
a. main conduit for microstructures which:
i. can be made porous for use with a non-porous housing, or
ii. itself can be made non-porous, and
iii. has flared ends for facilitated entry of the microstructure.
b. umbilical port, or ports, for the purpose of applying suction.

2. A surrounding housing or suitable covering for said porous connecting device to include an umbilical port for the purpose of achieving a negative gauge pressure within said housing

3. A method of utilization for said device to include:
a. Suctioning or negative gauge pressure within the micro-connector to draw microstructures into said device
b. Suctioning or negative gauge pressure within the surrounding housing to draw microstructures up against the inner wall of the said connecting device
c. The use of a bio-adhesive to attach the microstructure to the inside of the connector.
